(19) United States
(12) Patent Application Publication
(10) Pub. No.: US 2020/0345316 A1
(43) Pub. Date: Nov. 5, 2020

(54) Method To Communicate The Status OF Predetermined Conditions Without The Need For Any Manual Intervention

(71) Applicant: Ronald Allen Noel, Clemmons, NC (US)

(72) Inventor: Ronald Allen Noel, Clemmons, NC (US)

(21) Appl. No.: 16/445,603

(22) Filed: Jun. 19, 2019

Related U.S. Application Data
(60) Provisional application No. 62/690,685, filed on Jun. 27, 2018.

(51) Int. Cl. A61B 5/00; A61B 5/11; G08B 21/02

(52) U.S. Cl. CPC A61B 5/747; A61B 5/1117; A61B 5/746; A61B 2505/07; A61B 5/681; G08B 21/02; A61B 2560/0214; A61B 5/002

(57) ABSTRACT

POM will reduce the time between a Wearer's traumatic or emergency event and the arrival of needed assistance. A watch-like Wearable contains sensors that will monitor four vital signs, Fall Notification, and Geo Fencing violations 24 hours a day. Maximums and Minimums of the Vital Signs will be established by the Wearer's Physician. Any violation will automatically notify the appropriate parties.

POM performs automatically with no interaction from the Wearer. Total response time between an event and the notification for assistance is less than one minute. The Wearer's Physician will establish the limits for the four vital signs; Respiration, Heart Rate, Oxygen Saturation, Blood Pressure after a period of wearing to establish baselines. Fall Notification and Geo Fencing violations are also monitored. The device is reliable and comfortable to wear. The Wearer can reset the process if there is a valid reason. The Wearer's Contact is notified if the ALERT is not reset by the Wearer. If the Contact does not respond to the ALERT, either a second Contact or a 24/7/365 Security Call Center Operator will be notified. Global Positioning Software will provide the location of the Wearer.

A person of any age or health status can utilize POM if they wish to do so.

When the human body experiences a traumatic or an emergency condition; lack of oxygen, blunt force, loss of blood, temperature variation from normal, etc. cessation of life will occur unless these conditions are corrected in a matter of minutes. In most cases, the longer the body continues to experience an adverse condition, the greater the risk of permanent damage or cessation of life. Many times, the person experiencing the adverse conditions is alone and not capable of contacting anyone for help.

In addition, the Life Expectancy of humans of both sexes is approaching 85 years, (see, Our World in Data/life Expectancy (2018)). In 2016, 8.5 percent of the world population (617 million) are aged 65 and above. As reported in “An Aging World: 2015” this percentage will grow to above 16% of the world's population (1.6 billion) by 2050. (see: National Institute of Health (NIH.gov/news-events/news-releases/worlds-older-population-grows-dramatically, (2016)). Many of these individuals make the decision to live independently rather than be cared for in a location outside their current residence.

This system and method will monitor a Wearer's vital signs such as Heart Rate, Respiration, Oxygen Saturation, Blood Pressure, along with Fall Indication, and Geo Fencing capability via appropriate sensors embodied in a wearable device. Baseline values of these parameters will be established after a review of a period of use of the device. When any of the monitored parameters violate an established baseline, the Wearer will be notified. If the Wearer is capable and not in a traumatic or emergency event, they can reset the system. If the Wearer does not reset the system, an ALERT notification will be sent to a preestablished First Contact person. The Contact will check with the Wearer. If this check is not completed, the Contact will call appropriate numbers such as 911. The GPS coordinates from the Wearable or paired cellular phone will provide the location of the Wearer. If the First Contact does not respond to the ALERT, the system will notify a Second Contact or a 24/7/365 Call Center. After a verification call to the Wearer receives no response, the appropriate emergency agency will be notified. Again, the GPS coordinates will be available for the location of the Wearer. This system is valuable to a person of any age and any medical or physical condition.

Sixty-five percent of older adults with long-term care needs rely exclusively on family and friends to provide assistance. Another 30 percent supplement family care with paid assistance. Care provided by family and friends can determine whether an older person can remain at home. In fact, 50 percent of the elderly who have a long-term care need but no family available to care for them are in nursing homes, while only 7 percent who have a family caregiver are in institutional settings.

Military personnel can utilize POM to recover from various medical conditions. Healthcare Institutions will be able to automatically review the vital sign status of recently discharged patients. Setting the maximums and minimums to desired levels, the ALERT will be able to notify the Healthcare Institution of the status of the patient.

Adults and/or children who want to feel secure that they can notify a Contact when needed can utilize POM.

As of 2011, some 43.5 million adult family caregivers were taking care of someone 50+ years of age. Of these, 14.9 million were caring for someone with dementia. As more people live long enough to experience multiple health issues and dependency, more relatives will be facing this responsibility.

Living with Limitations

In 2005, 56 percent of persons age 80+ reported a severe disability and 29 percent of the 80+ population reported needing assistance.

In 2009, 25 percent of Medicare beneficiaries age 65+ reported difficulty with at least one Activity of Daily Living, (ADL). Physical limitations increase with age. Among men age 65-74, 13 percent reported being unable to perform at least one ADL, compared with 40 percent of men age 85+. For women, 19 percent age 65-74 were unable to perform at least one activity, compared with 53 percent of those age 85+.

Living Alone

Of the older adults who were living outside nursing homes or hospitals in 2010, nearly one third (11.3 million) lived alone. Older women are twice as likely as older men to live alone (37 percent and 19 percent, respectively). In 2010, 72 percent of older men lived with a spouse, only 42 percent of older women did.

Living arrangements differ by race and ethnicity. Older non-Hispanic White women and Black women are more likely than women of other races to live alone (39 percent each, compared with about 21 percent of older Asian women and 23 percent of older Hispanic women).

The likelihood of living alone increases with age. Among women age 75+, almost half (47 percent) lived alone in 2010.

(See: Institute On Aging Information on Senior Citizens Living in America, (2018))

If individuals elect to live along, they place themselves at greater risk of severe injury since they have no one to help if an adverse condition arises. POM, when used properly, can be the helping hand to notify others of the situation and request help. Every minute that is saved when help is needed, could be the difference between permanent disability or death and complete recovery.

### Method to Communicate the Status of Predetermined Conditions without the Need for any Manual Intervention

### SUMMARY

Inspired by a tragic event when a 40-year-old nurse, had a heart attack while alone in her kitchen and is now alive with severe brain damage, POM can be available to any age group. However, we agree with one Director of Senior Services that this will be a great tool for their members. The Director has volunteered to have their administrative staff participate in the pilot. After the process is tested, they wish to provide POM, at no cost, to 200 of their members on Medicaid especially those living alone.

Peace of Mind (POM) is a process that pairs a Wearable Smartwatch device with a Smartphone. The Wearer, the Contact person, and the Doctor of the Wearer determine the high and low readings for Oxygen Saturation, Blood Pressure, Respiration, and Pulse. Once these readings are placed into the POM Smartphone application of the Wearer, they will be monitored 24 hours a day when the Smartwatch is warn. If one of the Vital Signs exceeds the established limits, POM will alert the Wearer. If there is a valid reason, the Wearer can take steps to turn off and reset the process. If the Wearer is in distress, they can call the Contact or call 911. If the Wearer is not able to perform these functions, POM automatically calls the number of the Contact. The Contact will call the Wearer to investigate. If the Wearer cannot be reached, the Contact person can call 911. If the Contact person is not able to take the call from the Wearer, the call is automatically sent to a 24/7/365 Security Call Center. The Security Call Center Operator will attempt to contact the Wearer and if no answer, will call 911. The Global Positioning System, (GPS) of the Smartphone or Smartwatch will help locate the Wearer.

POM is being developed to assist all who wish to participate, with a process that will be able to assist them in obtaining help without any manual intervention. The parameters of the tracked vital signs will be set by the Wearer, the Contact and the Wearer's Doctor. POM will send an ALERT to the appropriate person to ensure help will arrive in the shortest period-of-time. Quality, Comfort, Durability, Repeatability, and Response Speed are the main goals for POM. A process that causes false readings or fails to communicate, during acceptable conditions of use, will cause customer frustration and they will spread that frustration though out the market.

### DESCRIPTION OF THE FIGURES

FIG. 1: Wearer to Contact only

FIG. 2: 24/7/365 coverage by Central Station

FIG. 3: 24/7/365 coverage with control panel connected to Central Station.

### DETAILED DESCRIPTION OF EMBODIMENTS OF THE INVENTION

The present invention will now be described more fully hereafter. This invention may, however, be embodied in different forms and should not be construed as limited to the embodiments set forth herein. Rather, these embodiments are provided so that this disclosure will be thorough and complete, and will fully convey the scope of the invention to these skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicated otherwise.

Unless otherwise defined, all terms (including technical and scientific terms) used herein have the same meaning as commonly understood by one of ordinary skill in the art to which this invention belongs. It will be further understood that terms such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limited of the invention. All publications, patent applications, patents, and other references mentioned herein are incorporated by reference in their entirety.

Also, as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, the specification states that a complex comprises compounds A, B, and C, it is specifically mentioned that any of A, B, or C, or a combination thereof can be omitted and disclaimed singularly or in any combination.

The present invention can be implemented in combination with cellular telephone, wearable medical measurement devices, centralized 24/7/365 security monitoring operations, and computer technology. Such systems may be implemented in accordance with known techniques or variations thereof that will be apparent to those skilled in the art.

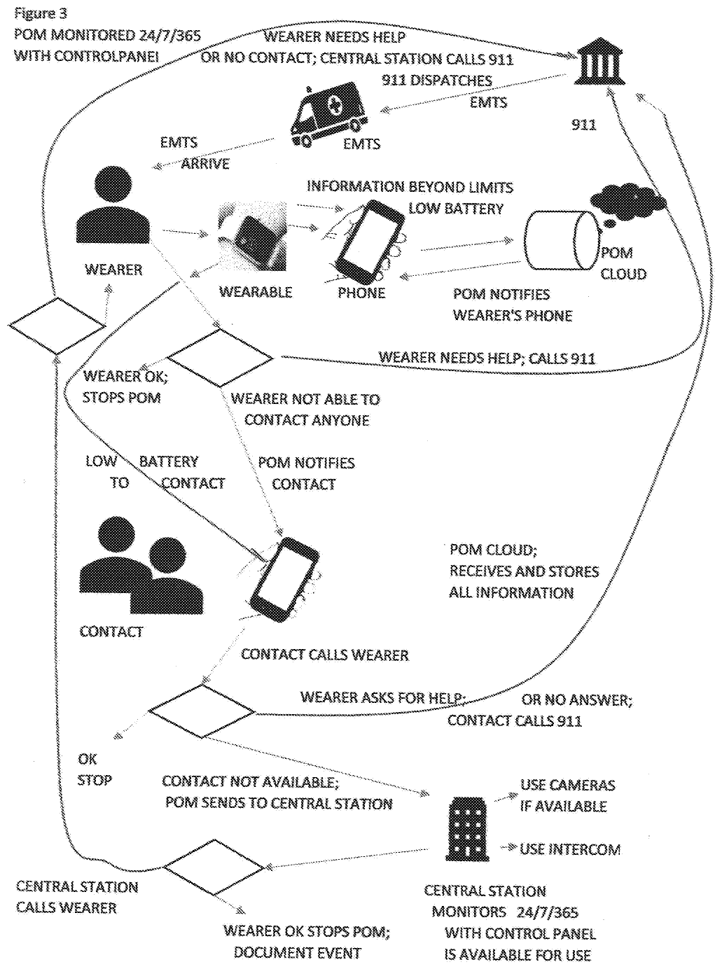

An example system of the present invention is schematically illustrated in FIG. 3. The system consists of these component items:
Wearable monitor of bodily functions
Cellular Phone for the Wearer
Cellular Phone for the Contact
Cellular Phone Network Provider
POM Cloud Platform
Monitoring Operations Facility
Control Panel in Wearer's place of residence
Emergency Call Center (911)
EMS Response Organization

The vital signs or characteristics of the body that will be reported by the wearable monitor will include as many of the following that are provided accurately. Accuracy can be verified when compared to calibrated healthcare devices:
Pulse Rate
Oxygen Saturation (SpO2)
Respiratory Rate
Blood Pressure
Fall indication; accelerometer
Motion or lack thereof
Geo Fencing
Note: This data will be integrated into the POM system using the most accurate sensor methods. As other indicators improve, data from these sensors will be incorporated into POM.

Additional Features may Include:
Display of daily Step number
Display of calorie used
Low battery indication
Data storage Function
Stored data can be wirelessly uploaded to mobile terminal
Clock function
Charging function

### Preparation to use POM

When a Wearer candidate decides to incorporate POM into their life, they will first need to identify a Contact. This is a person who the Wearer trusts will be responsible to function as the first contact when POM indicates that one or more of the Wearer's vital signs is/are currently higher or lower than the maximum and minimum settings as established for the Wearer, the Fall indicator is activated, or the Geo Fence is violated, and the Wearer is not capable of making contact with an Emergency Call Center (911).

The Wearer and the Contact will be encouraged to review the information provided to educate them both on the advantages and the limitations of POM. They will be able to follow the process to set the maximum and minimum values for the situation being tracked. Options for wearable items, compatible smartphones, and the cellular phone network provider offering the best cellular service in the area will be listed. They will be instructed how to recharge the battery of their selected wearable and what to do if the unit may have to be removed for any reason.

In addition, they will be invited to sign a waiver that will hold harmless the provider and the communication organization. POM is only an indicator that warns when a tracked function is above or below a set function. If that function is not set properly for the environment of the Wearer, a false reading may be provided. An example is blood saturation. When the Wearer is in an environment that has fewer air particles, such as in an aircraft or on a mountain top at high altitude, the amount of air in the blood (SpO2) may decrease. This may violate the tolerance set for that vital sign. The Wearer and Contact will be encouraged to have the vital sign readings of the wearable compared to Medical Center equipment when the Wearer has an appointment with their Healthcare Practice Provider. A Physician or Specialist may wish to have a specific vital sign set at specific levels due to the Wearer's physical conditions.

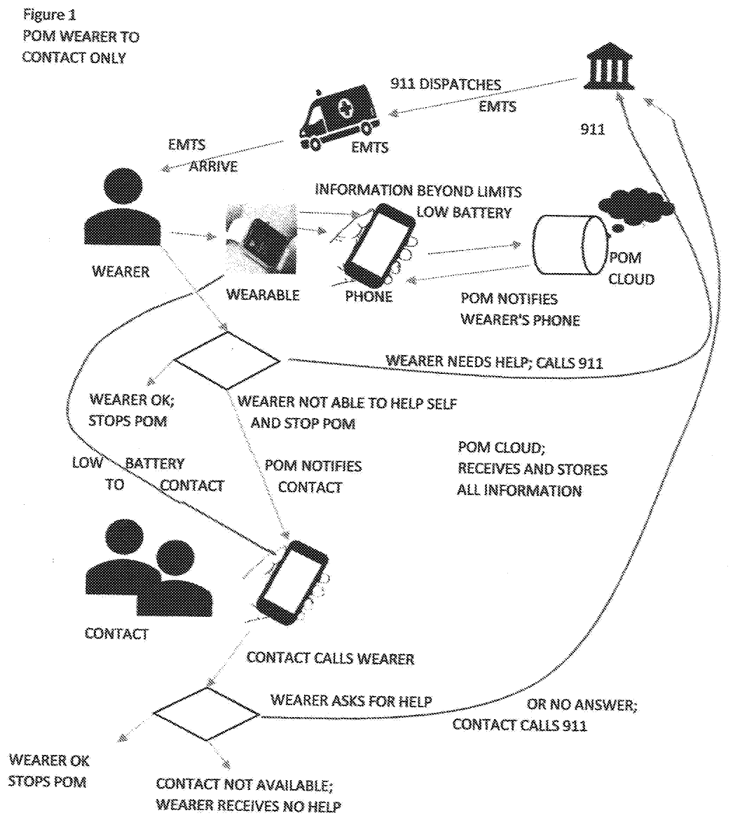

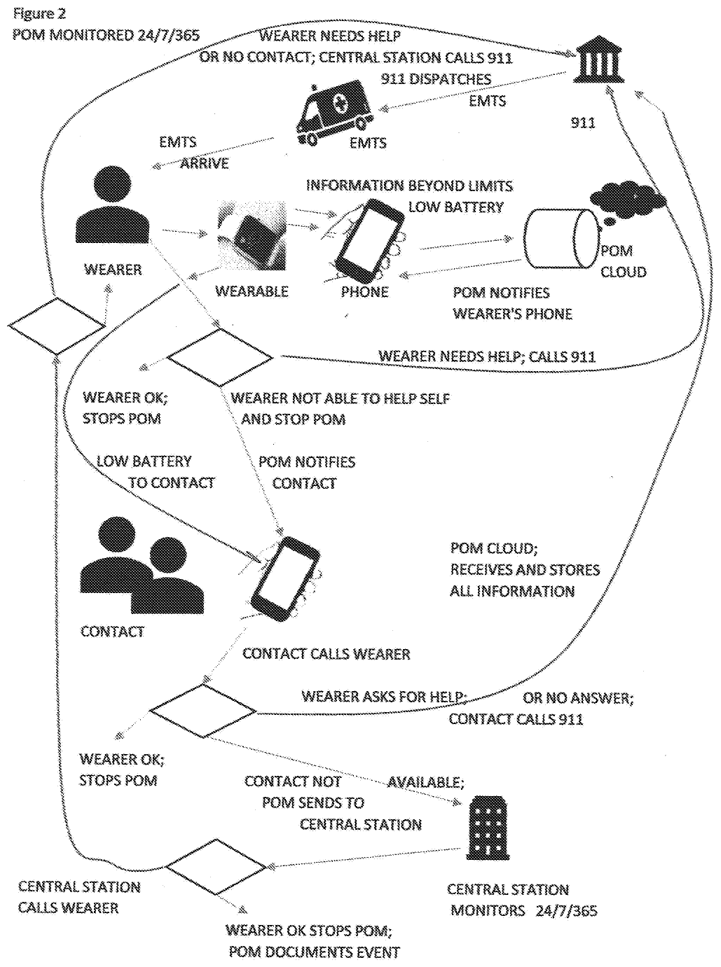

The Wearer and Contact will have to determine if they what one of three options:
1. Wearer to Contact Only; FIG. 1
2. 24/7/365 coverage by Central Station; FIG. 2
3. 24/7/365 coverage with control panel of Central Station; FIG. 3

Once the Wearer and Contact have reviewed the process of POM, have become familiar with the wearable and cellular smartphone to which it is paired, have determined the option for POM, and signed the hold harmless agreement and contract, they are ready to request the password for the establishment of the maximum and minimum of each vital sign during regular or anticipated activity or lack thereof. The wearable will be worn over the next 2 to 4 weeks to establish the maximum and minimum values of each vital sign. This data, once gathered, will be provided to the Wearer's Healthcare Practice Provider for review and for recommendations for the settings of each vital sign. A printout of the settings will be signed by the Physician after the values have been established. Payment of the monthly charge will be due one month in advance on a specific date of the month. If two successive payments are not forwarded by the due date, the Wearer, Contact, and Physician will be contacted by certified letter indicating the date the POM system will be cancelled.

### Wearer to Contact Only See FIG. 1.

The appropriate APPLICATION will now be loaded into the Wearer's smartphone and the Contact's smartphone. This option of POM can be provided to the Wearer and Contact for the most modest monthly charge. This process will rely totally on the Wearer and the Contact for operation and attention to the ALERTS when the limits are exceeded.

Before POM is officially released for the Wearer to Contact Only system, the Wearer and Contact will be asked to test the system. This test will require artificial overages of vital sign tolerance. The artificial overage will be caused by the removal of the wearable. The first test will be answered by the Wearer. They will be able to call their Contact, a Physician or call 911 with no intervention from the Contact. When the Wearer answers the ALERT, this portion of the test will STOP. The POM test system will automatically restart after the wearable is returned to the body of the Wearer. The next test will be initiated by the removal of the wearable. This time the Wearer will allow the alarm to flow to the Contact's smartphone. The Contact will answer the ALERT. The Contact will call the Wearer's smartphone. As contact with the Wearer is completed, the Wearer and Contact will verify that the contact was made. The POM test system will automatically restart after the wearable is returned to the body of the Wearer. The next test will be initiated by the removal of the wearable. Again, the Wearer will allow the alarm to flow to the Contact's smartphone. The Contact will answer the ALERT. The Contact will call the Wearer's smartphone. The Wearer will not answer the call. If contact with the Wearer is not completed, the Contact will enter a unique password to convert the two smartphones to an intercom system. The Wearer and the Contact will verify the intercom function is operational. The POM test process will STOP when the wearable is returned to the Wearer's body. The POM test system will automatically restart after the wearable is returned to the body of the Wearer. If this were a real situation, the Contact will make the decision to contact the Wearer or 911.

During the operation of POM, if the Contact does not make contact with the Wearer or if contact was made and the Wearer required assistance, the Contact would call 911. If the Wearer could not provide the location., the GPS of the Wearer's phone will be used to provide the location of the Wearer. This information will be provided to the EMS responders.

### POM 24/7/365 See FIG. 2

If the Wearer and Contact wish to provide 24/7/365 coverage by a Third-Party Monitoring Organization, this option will require a different password, possibly different smartphones and a different wearable, plus a higher monthly charge. Payment of the monthly charge will be due one month in advance on a specific date of the month. If two successive payments are not forwarded by the due date. The Wearer, Contact, and Physician will be contacted by certified letter indicating the date the POM system will be cancelled.

Before POM is officially connected to the POM 24/7/365 monitoring system, the Wearer and Contact will be asked to test the system. This test will require artificial overage of vital sign tolerance. The artificial overage will be caused by the removal of the wearable. The first test will be answered by the Wearer. The Wearer will be able to call their Contact, a Physician, or call 911 with no intervention from the Contact or Monitoring Organization Operator. When the Wearer makes a phone call to the Contact, even if the call is not answered, the call will STOP the POM test system. The POM test system will automatically restart after the wearable is returned to the body of the Wearer. The next test will be initiated by the removal of the wearable. This time the Wearer will allow the alarm to flow to the Contact's smartphone. The Contact will answer the ALERT and call back to the Wearer. This will indicate to the Wearer that the Contact has been notified by POM. If this were a real situation, the Contact will make the decision to contact the Wearer or 911. The POM test process will STOP when the Contact answers the ALERT. POM will automatically restart after the wearable is returned to the body of the Wearer. The next test will be initiated by the removal of the wearable. Again, the Wearer will allow the alarm to flow to the Contact's smartphone. The Contact will answer the ALERT. The Contact will call the Wearer's smartphone. The Wearer will not answer the call. If contact with the Wearer is not completed, the Contact will enter a unique password to convert the two smartphones to an intercom system. The Wearer and the Contact will verify the intercom function is operational. The POM test process will STOP when the wearable is returned to the Wearer's body. The POM test system will automatically restart after the wearable is returned to the body of the Wearer. If this were a real situation, the Contact will make the decision to contact the Wearer or 911. For the final test, a special PASS CODE will be provided for contact to the Monitoring Organization Operator. This will indicate to the Monitoring Organization Operator that this is a test of the system. A specified number of these tests will be allowed per specified period. This final test will be initiated by the removal of the wearable. This time the Wearer and Contact will allow the ALERT to flow through to the Monitoring Organization Operator. Once the ALERT reaches the Monitoring Organization Operator, the operator will check the data sent by the Wearer's device. The Operator will verify that their functions for answering, receiving data, GPS of the Wearer's Smartphone, and being provided a contact number is complete.

Upon the completion of this check of the POM system, the Monitoring Organization Operator will call the Wearer for verification. The test instructions will advise the Wearer to answer the phone call. When the Wearer answers, the test will STOP. The POM test system will automatically restart after the wearable is returned to the body of the Wearer. During the operation of POM, if the Monitoring Organization Operator does not make contact with the Wearer or if contact was made and the Wearer required assistance, the Operator would call 911. If the Wearer could not provide their location, the GPS of the Wearer's phone will be used to provide the location of the Wearer. This information will be provided to the EMS responders.

### POM 24/7/365 with Control Panel See FIG. 3

If the Wearer and Contact wish to provide 24/7/365 With Control Panel coverage by a Third-Party Monitoring Organization, this option will require a different password, possibly different smartphones and a different wearable, and a security Control Panel installed in the residence of the Wearer, plus a higher monthly charge. Payment of the monthly charge will be due one month in advance on a specific date of the month. If two successive payments are not forwarded by the due date. The Wearer, Contact, and Physician will be contacted by certified letter indicating the date the POM system will be cancelled.

Before POM is officially connected to the POM 24/7/365 With Control Panel monitoring system, the Wearer and Contact will be asked to test the system. This test will require artificial overage of vital sign tolerance. The artificial overage will be caused by the removal of the wearable. The first test will be answered by the Wearer. They will be able to call their Contact, a Physician, or call 911 with no intervention from the Contact or Monitoring Organization Operator. When the Wearer makes a phone call to the Contact, even if the call is not answered, the call will STOP the POM test system. The POM test system will automatically restart after the wearable is returned to the body of the Wearer. The next test will be initiated by the removal of the wearable. This time the Wearer will allow the alarm to flow to the Contact's smartphone. The Contact will answer the ALERT and call back to the Wearer's smartphone. This will indicate to the Wearer that the Contact has been notified by POM. If this were a real situation, the Contact will make the decision to contact the Wearer or 911. The POM test process will STOP when the Contact answers the ALERT. POM will automatically restart after the wearable is returned to the body of the Wearer. The next test will be initiated by the removal of the wearable. Again, the Wearer will allow the alarm to flow to the Contact's smartphone. The Contact will answer the ALERT. The Contact will call the Wearer's smartphone. The Wearer will not answer the call. If contact with the Wearer is not completed, the Contact will enter a unique password to convert the two smartphones to an intercom system. The Wearer and the Contact will verify the intercom function is operational. The POM test process will STOP when the wearable is returned to the Wearer's body. The POM test system will automatically restart after the wearable is returned to the body of the Wearer. If this were a real situation, the Contact will make the decision to contact the Wearer or emergency services. For the next test, a special PASS CODE will be provided for contact to the Monitoring Organization Operator. This will indicate to the Monitoring Organization Operator that this is a test of the system. A specified number of these tests will be allowed per specified period. The test will be initiated by the removal of the wearable. This time the Wearer and Contact will allow the ALERT to flow through to the Monitoring Organization Operator. Once the ALERT reaches the Monitoring Organization Operator, the operator will check the data sent by the Wearer's device. The Operator will verify that their functions for answering, receiving data, GPS of the Wearer's smartphone, and being provided a contact number is complete. Upon the completion of this check of the POM system, the Monitoring Organization Operator will call the control panel. The Monitoring Organization Operator will activate the Intercom System. The Wearer and the Monitoring Organization Operator will have a conversation with the Wearer in various locations in the residence. The cameras in the residence will also be checked for functionality during this test. The test will STOP when the wearable is returned to the body of the Wearer. The POM test system will automatically restart after the wearable is returned to the body of the Wearer.

During the operation of POM, if the Monitoring Organization Operator does not make contact with the Wearer or if contact was made and the Wearer required assistance, the Operator would call 911. If the Wearer could not provide their location, the GPS of the Wearer's phone will be used to provide the location of the Wearer. This information will be provided to the EMS responders. If the camera has identified the location of the Wearer, the information will be provided to EMS.

### Wearer Self Initiation of POM

The POM APPLICATION on the Wearer's smartphone has a method to set the process in motion if they need assistance. The same steps as the automatic POM system will be placed into motion. The removal of the wearable without the notification from the Wearer, will initiate the POM system.

### Wearer Self Interruption of POM

In the event that POM will have to be turned off, the Wearer or the Contact can suspend POM for a specified period. There is a method on the APPLICATION in the Wearer's phone and the Contact's phone that will allow POM to be removed from the communication process. This off-line time can be entered in periods of 60 minutes. During this off-line period, a reminder text or e-mail will be sent to the Wearer and to the Contact at the end of every 60-minute period. Once all off-line periods have expired, POM will automatically return to an on-line condition. A text or e-mail will be sent to the Wearer and Contact indicating that POM is back on-line. If POM has been offline for more than 2 hours after a scheduled off-line period, POM will send a message to the

Wearer and the Contact. Upon receipt of this alarm, the Monitoring Organization Operator will verbally connect with the Wearer and Contact. If this connection is not made to either of the two, the Monitoring Organization Operator will notify 911. Notification by text or e-mail will be sent to both from the Monitoring Organization Operator the Wearer and the Contact indicating that 911 has been dispatched.

### Initiation of POM

If all situations listed above functioned properly and all Wearer and Contact information is complete and accurate, POM is ready to maintain surveillance of the Wearer. After the initial payment has been received by a POM executive, POM will start automatically.

### Operation of POM All Three Options

POM is an automatic method to send the Wearer's vital signs to the appropriate person in an effort to notify the appropriate person that the Wearer is in need of help. If the Wearer cannot contact help personally, POW will provide a method to solicit help with no Wearer intervention. The purpose of POM is to relay the vital signs of the Wearer to the person(s) in the process when the Wearer's vital signs exceed the settings established by the Wearer, the Contact, and the Wearer's Physician. POM is intended to reduce the time needed to provide help to the Wearer. Hopefully, this help will provide timely assistance to the Wearer so that minimal medical impact will be felt by the Wearer.

POM will use a Wearable that transmits the Wearer's vital signs to the Wearer's Smartphone and the POM application. The POM system will review the vital signs data from the measurement device to evaluate the vital signs of the Wearer and compare them to the maximum and minimum settings of each selected vital sign. The Wearer, Contact, and Physician can select the features and the settings they wish to monitor. Next, they select the Smartphone and Cellular Network Provider that is available and most reliable for the geographic area near the Wearer's residence. They will need to know the characteristics of the POM system and how to react to the warning signs. This will allow the Wearer to contact the appropriate party with or without their intervention for help if they are able or unable to make contact. After selecting their Contact person, their wearable instrument, smartphone and cellular network provider for themselves and for the Contact, they will perform a 2 to 4 weeks wearing of the instrument to determine the range of the selected vital signs. After consultation with their Healthcare Provider, the vital sign parameters will be entered into the POM system.

The wearable instrument will monitor the selected vital signs. If one or more of these vital signs, exceed the preset maximum or minimum, the Fall Indicator is activated, or the Geo Fencing footprint is violated, the POM system will start the communication process.

In a preferred embodiment, components of the system are suggested to be a wearable instrument that is waterproof, has long lasting batter life and is easily recharged. The instrument will accurately provide the vital signs that are suggested to be tracked by the Physician for the Wearer. The wearable will need to transfer current data to the Wearer's smartphone (Android or iPhone) and be able to be managed by the POM Application. The Cellular Network Provider will be selected to provide the best service and coverage in the usual geographic area traveled by the Wearer. POM will not function properly if there is no connection to the Smartphone and to the Cellular Service Provider network. The Contact person will also have to select a smartphone that will manage the POM process. Again, the Cellular Service Provider will need to be carefully selected. POM will not be able to function properly if the smartphone service is not robust.

### Operation of POM Wearer to Contact Only See FIG. 1.

The maximum and minimum settings of the Wearer's vital signs have been entered into POM. If one or more vital signs are found to be beyond the established maximum or minimum, the Fall Indicator is activated, or the Geo Fencing footprint is violated, POM will react and advise the Wearer. The Wearer can react to the ALERT and cancel at that point, then call their Contact person or 911 for help if needed.

If the ALERT is ignored by the Wearer and allowed to flow as designed, it will send the ALERT to the Contact person. If the Contact person reacts to the ALERT, they can cancel the ALERT and determine if they should contact the Wearer or contact 911 to provide help. The location of the Wearer will be provided by the GPS location of the Wearer's smartphone. This geographic location information will be provided to EMS.

This will end the Wearer to Contact Only option of POM.

### Operation of POM POM 24/7/365 See FIG. 2.

The maximum and minimum settings of the Wearer's vital signs have been entered into POM. If one or more vital signs are found to be beyond the established maximum or minimum, the Fall Indicator is activated, or the Geo Fencing footprint is violated, POM will react and advise the Wearer.

The Wearer can react to the ALERT and cancel at that point, then call their Contact person or 911 for help if needed.

If the ALERT is ignored by the Wearer and allowed to flow as designed, it will send the ALERT to the Contact person. If the Contact person reacts to the ALERT, they can cancel the ALERT and determine if they should contact the Wearer or contact 911 to provide help. The location of the Wearer will be provided by the GPS location of the Wearer's smartphone. This geographic location information will be provided to EMS

If the Contact person does not react to the ALERT and answer the call, the ALERT will pass to the Monitoring Organization Operator. When the Monitoring Organization Operator receives the call from POM they will be provided with the Identification about the Wearer, the Contact person and the Physician listed. They will also be provided with the Wearer's location from the GPS of the Wearer's smartphone, along with the current vital sign, fall indicator, or Geo Fence violation. At that point, the Operator will phone the Wearer. If the Wearer can be contacted, the Wearer will be asked what help is needed. The Operator can call 911 or the Contact person. The location of the Wearer will be provided by the GPS in the Wearer's phone. The Operator will be able to provide all information to the EMS staff.

To close the final loop in the situation, the Operator will contact the Wearer and Contact person via text or e-mail and request a response.

After a full event, it is suggested that the Wearer and Contact person retest the POM system to ensure it is reset and available.

### Operation of POM POM 24/7/365 with Control Panel See FIG. 3.

The maximum and minimum settings of the Wearer's vital signs have been entered into POM. If one or more vital signs are found to be beyond the established maximum or minimum, POM will react and advise the Wearer. The Wearer can react to the ALERT and cancel at that point, then call their Contact person or 911 for help if needed. If the ALERT is ignored by the Wearer and allowed to flow as designed, it will send the ALERT to the Contact person. If the Contact person reacts to the ALERT, they can cancel the ALERT and determine if they should contact the Wearer or contact 911 to provide help. The location of the Wearer will be provided by the GPS location of the Wearer's smartphone.

If the Contact does not communicate with the Wearer, the Contact will call 911. The GPS system of the Wearer's smartphone will be used to identify the location of the Wearer. This geographic location information will be provided to EMS

If the Contact person does not react to the ALERT and answer the call, the ALERT will pass to the Monitoring Organization Operator. When the Monitoring Organization Operator receives the call from POM they will be provided with the Identification of the Wearer, the Contact person and the Physician listed. They will also be provided with the Wearer's location from the GPS of the Wearer's smartphone, along with the current vital sign, fall indicator, or Geo Fence violation. At that point, the Operator will phone the Wearer. If the Wearer can be contacted, the Wearer will be asked what help is needed.

If the Wearer can not be contacted, the Monitoring Organization Operator will activate the intercom system on the control panel. The Operator will try to contact the Wearer. If the Wearer can be contacted, the Wearer and the Monitoring Organization Operator will have a conversation about the condition of the Wearer. If cameras are included with this option, the Operator can review the camera images to provide further information about the Wearer. This will only be functional if the Wearer is inside the covered residence.

The Operator can call 911 or the Contact person. The location of the Wearer will be provided by the GPS in the Wearer's phone. The Operator will be able to provide all information to the EMS staff.

To close the final loop in the situation, the Operator will contact the Wearer and Contact person via text or e-mail and request a response. After a full event, it is suggested that the Wearer and Contact person retest the POM system to ensure it is reset and available.

Glossary:

Wearer: The person who is wearing the device(s) which is monitoring the body conditions listed below.

Wearable: The unit that contains the sensors that are used to capture and transmit the data to the application in the wearer's phone

Wearer's Smart Phone: The smart phone that is the first contact from the wearable. It contains the data and the application that provides the interpretation of the data and forwards the phone call to the contact's phone when the designated data is in the range identified to be critical to the safety of the wearer.

Contact: The person who is designated to receive the initial contact from the wearer's smart phone.

SC: Security Central The organization that manages the 24/7/365 monitoring of the alerts and takes the appropriate action.

POM CLOUD: Where the information is received, managed and acted upon.

Oxygen Saturation: The estimated amount of Oxygen contained in the Red Blood Cells. The measurement method SpO2 stands for peripheral capillary Oxygen saturation and measures the percentage of oxygenated hemoglobin of the blood.

A value of 95% to 100% is considered satisfactory. If a value is below 95%, it is a sign of poor blood oxygenation or hypoxia.

ADLs: Activities of Daily Living; eating, bathing, dressing, toileting, walking, and continence.

What is claimed is:

1. Function of POM
POM is an automatic method of tracking the vital signs, falls, and Geo Fencing selected for the Wearer. These vital signs and conditions will be transferred to a Smartphone via BLE Technology within less than 30 seconds. The data is saved, and POM will have the ability to provide time and date of trending data for the selected vital signs.
The Wearer will select a Contact person who will be the First Contact in the POM system.
a. After an initial test period, a maximum and minimum of the Wearer's vital signs will be established by the Wearer, the Contact person and the Physician of record for the Wearer.
b. If a maximum or minimum of a vital sign is exceeded or a fall or Geo Fence violation is experienced, POM will provide an ALERT to the Wearer.
c. The Wearer will be able to intercept the ALERT and notify the Contact person or 911 if help is needed. If help is not needed, the Wearer can reset the POM system. A notification of the situation will be saved in the data of POM.
d. The location of the Wearer will be available from the Smartphone or the Wearable of the Wearer.
e. The Wearable and Smartphone will indicate Low Battery Status to the Wearer and to the Contact.

2. If the ALERT is not reset by the Wearer due to the situation, the ALERT will be passed through to the Contact person. POM will provide information about the status of the Vital Sign overage, Fall, or Geo Fence violation of the Wearer.
a. The Contact person can react to the ALERT and restart the POM process without it going to further processes.
b. The Contact person can call the Wearer's Smartphone or Wearable to determine the situation. If needed, the Contact person can intercede or call 911.
c. The Contact person will be provided with the details of the situation from the Wearable device so this information can be provided to the EMS staff.
d. The GPS of the Wearer's smartphone or Wearable will provide the location of the Wearer.
e. The Contact will have to ensure they are available 24/7/365 so no ALERTS are missed.
f. The Contact will be responsible for contacting 911 or other help as needed.
g. An additional Contact person can be added to the system if desired.

3. Adding a 24/7/365 Monitoring Organization to POM
By adding the telephone number for a 24/7/365 Monitoring Organization as the third Contact total coverage can be available. If the Contact person is unavailable, POM will allow the ALERT to pass to the next step and notify a 24/7/365 Monitoring Organization Operator.
a. The Monitoring Organization Operator will be provided with detailed information about the Wearer: Smartphone or Wearable number, address, GPS location, Contact person, Contact person's phone number, and Physician of record with a phone number.
b. Upon receiving the ALERT, the Operator will call the Wearer. If contact is made and the Wearer can discuss the situation, the Wearer will be asked if help is needed. If the Wearer cannot provide
c. an appropriate answer, the Monitoring Organization Operator can inform the Contact person, 911, or dispatch EMS staff.
d. If the Wearer can provide an appropriate answer that help is not needed, the Monitoring Organization Operator will be able to send a text or e-mail to the Wearer, Contact, and Physician to document the situation.
e. POM will reset.
f. By adding a Security System to the home, intercoms, electronic locks, and cameras can be utilized if the Wearer is within the area of the Security System.
